(19) United States
(12) Patent Application Publication
(10) Pub. No.: US 2007/0093899 A1
(43) Pub. Date: Apr. 26, 2007

(54) Apparatus and methods for treating bone

(76) Inventors: Christof Dutoit, Solothurn (CH); Andreas Appenzeller, Biel (CH); Thierry Stoll, Meinisberg (CH); Alfred Benoit, Lengnau (CH); Stefan Mathys, Basel (CH); Simon Stucki, Bern (CH)

Correspondence Address: JONES DAY, 222 EAST 41ST STREET, NEW YORK, NY, 10017-6702, US

(21) Appl. No.: 11/527,280

(22) Filed: Sep. 25, 2006

Related U.S. Application Data
(60) Provisional application No. 60/725,773, filed on Oct. 12, 2005.
(60) Provisional application No. 60/728,442, filed on Oct. 19, 2005.
(60) Provisional application No. 60/730,909, filed on Oct. 27, 2005.
(60) Provisional application No. 60/733,026, filed on Nov. 3, 2005.
(60) Provisional application No. 60/722,064, filed on Sep. 28, 2005.
(60) Provisional application No. 60/726,835, filed on Oct. 13, 2005.
(60) Provisional application No. 60/733,647, filed on Nov. 4, 2005.
(60) Provisional application No. 60/753,782, filed on Dec. 23, 2005.
(60) Provisional application No. 60/789,956, filed on Apr. 5, 2006.
(60) Provisional application No. 60/748,377, filed on Dec. 8, 2005.

(51) Int. Cl. A61F 2/44

(52) U.S. Cl. 623/17.11

(57) ABSTRACT

Implants and methods for minimally invasive augmentation and repositioning of vertebrae may comprise one or more expandable members, e.g., stents, implants, surrounding a balloon-tipped catheter or other expansion device, inserted into a vertebral body or other bone. Expansion of the expandable member within the vertebral body or other bone may reposition the fractured bone to a desired height and augment the bone to maintain the desired height. A bone cement or other filler can be added to further augment and stabilize the vertebral body or other bone.

### CROSS-REFERENCE TO RELATED APPLICATIONS

The present application claims priority to U.S. Provisional Application Nos. 60/725,773 filed Oct. 12, 2005; 60/715,188 filed Sep. 8, 2005; 60/728,442 filed Oct. 19, 2005; 60/730,909 filed Oct. 27, 2005; 60/733,026 filed. Nov. 3, 2005; 60/722,064 filed Sep. 28, 2005; 60/726,835 filed Oct. 13, 2005; 60/733,647 filed Nov. 4, 2005; 60/753,782 filed Dec. 23, 2005; 60/789,956 filed Apr. 5, 2006; and 60/748,377 filed Dec. 8, 2005, and U.S. patent application Ser. No. 11/471,169 filed on Jun. 19, 2006.

### FIELD OF THE INVENTION

The invention relates to surgical implants, and more particularly to minimally invasive apparatus and methods for augmenting bone, preferably vertebrae and/or restoring spinal lordosis.

### BACKGROUND OF THE INVENTION

Vertebral compression fractures, as illustrated in FIG. 1, represent a generally common spinal injury and may result in prolonged disability. F. Margerl et al: A comprehensive classification of thoracic and lumbar injuries, Eur Spine J 184-201, 1994. These fractures involve collapsing of one or more vertebral bodies 12 in the spine 10. Compression fractures of the spine usually occur in the lower vertebrae of the thoracic spine or the upper vertebra of the lumbar spine. They generally involve fracture of the anterior portion 18 of the affected vertebra 12 (as opposed to the posterior side 16). Spinal compression fractures can result in deformation of the normal alignment or curvature, e.g., lordosis, of vertebral bodies in the affected area of the spine. Spinal compression fractures and/or related spinal deformities can result, for example, from metastatic diseases of the spine, from trauma or can be associated with osteoporosis. Until recently, doctors were limited in how they could treat such compression fractures and related deformities. Pain medications, bed rest, bracing or invasive spinal surgery were the only options available.

More recently, minimally invasive surgical procedures for treating vertebral compression fractures have been developed. These procedures generally involve the use of a cannula or other access tool inserted into the posterior of the effected vertebral body through the pedicles. The most basic of these procedures is vertebroplasty, which literally means fixing the vertebral body, and may be done without first repositioning the bone.

Briefly, a cannula or special bone needle is passed slowly through the soft tissues of the back. X-ray image guidance, along with a small amount of x-ray dye, allows the position of the needle to be seen at all times. A small amount of polymethylmethacrylate (PMMA) or other orthopedic cement is pushed through the needle into the vertebral body. PMMA is a medical grade substance that has been used for many years in a variety of orthopedic procedures. Generally, the cement is mixed with an antibiotic to reduce the risk of infection, and a powder containing barium or tantalum, which allows it to be seen on the X-ray.

Vertebroplasty can be effective in the reduction or elimination of fracture pain, prevention of further collapse, and a return to mobility in patients. However, this procedure may not reposition the fractured bone and therefore may not address the problem of spinal deformity due to the fracture. It generally is not performed except in situations where the kyphosis between adjacent vertebral bodies in the effected area is less than 10 percent. Moreover, this procedure requires high-pressure cement injection using low-viscosity cement, and may lead to cement leaks in 30-80% of procedures, according to recent studies. Truumees, Comparing Kyphoplasty and Vertebroplasty, Advances in Osteoporotic Fracture Management, Vol. 1, No. 4, 2002. In most cases, the cement leakage does no harm. In rare cases, however, polymethymethacrylate or other cement leaks into the spinal canal or the perivertebral venous system and causes pulmonary embolism, resulting in death of the patient. J. S. Jang: Pulmonary Embolism of PMMA after Percutaneous Vertebroplasty, Spine Vol. 27, No. 19, 2002.

More advanced treatments for vertebral compression fractures generally involve two phases: (1) reposition, augmentation or restoration of the original height of the vertebral body and consequent lordotic correction of the spinal curvature; and (2) filling or addition of material to support or strengthen the fractured bone.

One such treatment, balloon kyphoplasty (Kyphon, Inc.), is illustrated in FIGS. 2A-D. A catheter having an expandable balloon tip is inserted through a cannula, sheath or other introducer into a central portion of a fractured vertebral body comprising relatively soft cancellous bone surrounded by fractured cortical bone (FIG. 2A). Kyphoplasty then achieves the reconstruction of the lordosis, or normal curvature, by inflating the balloon, which expands within the vertebral body restoring it to its original height (FIG. 2B). The balloon is removed, leaving a void within the vertebral body, and PMMA or other filler material is then injected through the cannula into the void (FIG. 2C) as described above with respect to vertebroplasty. The cannula is removed and the cement cures to fill or fix the bone (FIG. 2D).

Disadvantages of this procedure include the high cost, the repositioning of the endplates of the vertebral body are lost after the removal of the balloon catheter, and the possible perforation of the vertebral endplates during the procedure. As with vertebroplasty, perhaps the most feared, albeit remote, complications related to kyphoplasty are related to leakage of bone cement. For example, a neurologic deficit may occur through leakage of bone cement into the spinal canal. Such a cement leak may occur through the low resistance veins of the vertebral body or through a crack in the bone which had not been appreciated previously. Other complications include; additional adjacent level vertebral fractures, infection and cement embolization. Cement embolization occurs by a similar mechanism to a cement leak. The cement may be forced into the low resistance venous system and travel to the lungs or brain resulting in a pulmonary embolism or stroke. Also, the kyphon balloon is elastic and is not suited to expand a stent. Due to stent resistance, the kyphon balloon will expand anteriorly and posteriorly of the stent and suddenly explode when the stent borders cut the balloon. Additional details regarding balloon kyphoplasty may be found, for example, in U.S. Pat. Nos. 6,423,083, 6,248,110, and 6,235,043 to Riley et al.; Gantis et al., Balloon kyphoplasty for the treatment of pathological vertebral compression fractures, Eur Spine J 14:250-260, 2005; and Lieberman et al., Initial outcome and efficacy of Kyphoplasty in the treatment of painful osteoporotic vertebral compression fractures, Spine 26(14):1631-1638, 2001, each of which is incorporated by reference herein in its entirety.

Another approach for treating vertebral compression fractures is the Optimesh system (Spineology, Inc., Stillwater, Minn.), which provides minimally invasive delivery of a cement or allograft or autograft bone using an expandable mesh graft balloon, or containment device, within the involved vertebral body. The balloon graft remains inside the vertebral body after its inflation, which prevents an intraoperative loss of reposition, such as can occur during a kyphoplasty procedure when the balloon is withdrawn. One drawback of this system, however, is that the mesh implant is not well integrated in the vertebral body. This can lead to relative motion between the implant and vertebral body, and consequently to a postoperative loss of reposition. Additional details regarding this procedure may be found, for example, in published U.S. Patent Publication Number 20040073308, which is incorporated by reference herein in its entirety.

Still another procedure used in the treatment of vertebral compression fractures is an inflatable polymer augmentation mass known as a SKy Bone Expander. This device can be expanded up to a pre-designed size and Cubic or Trapezoid configuration in a controlled manner. Like the Kyphon balloon, once optimal vertebra height and void are achieved, the SKy Bone Expander is removed and PMMA cement or other filler is injected into the void. This procedure therefore entails many of the same drawbacks and deficiencies described above with respect to kyphoplasty.

A proposed improved procedure for repositioning and augmenting vertebral body compression fractures is vertebral body stenting, for example as described in Fürderer et al., “Vertebral body stenting”, Orthopäde 31:356-361, 2002; European Patent Application publication number EP1308134A3; and United States Patent Application publication number US2003/0088249, each of which is incorporated by reference herein in its entirety. Veterbral body stenting, as depicted for example in FIG. 3, generally involves inserting into a vertebral body a balloon-tipped catheter (e.g., such as a kyphoplasty balloon) surrounded by a stent (e.g., such as those used in angioplasty). After insertion of the balloon and stent, the balloon is inflated, e.g., using fluid pressure, thereby expanding the stent within the vertebral body. After expansion of the stent, the balloon may be deflated and removed, with the stent remaining inside the vertebral body in an expanded state to fill the vertebral body.

While the concept of vertebral body stenting provides promise over other known methods for treating compression fractures, there remains a need for improved stents and other expandable implants and related methods for repositioning and augmenting fractured vertebral bodies and other bones.

### SUMMARY OF THE INVENTION

The present invention provides apparatus and methods for minimally invasive augmentation of vertebral bodies. In one embodiment, the present invention provides an implant and method for correction of vertebral fractures and other disorders of the spine. For example, one or more stents or other expandable implants may be inserted into a vertebral body damaged by a vertebral compression fracture. As the one or more implants are inserted into a vertebral body and expanded, they may fill a central portion of the vertebral body and may push against the inner sides of the endplates of the vertebral body, thereby providing structural support and tending to restore the vertebra to its original height. Optionally, the one or more expandable implants may comprise a shape-memory alloy or other material that expands or changes configuration after implantation, which may lead to a thorough integration of the implant into the bone and/or help restore the height of the damaged vertebral body. After implantation, a bone cement (e.g., PMMA or tricalcium phosphate), bone chips, demineralized bone, or other filler material may be added to aid in stabilizing the bone and securing the implant in place within the bone.

The stents or other expandable implants may be comprised of any biocompatible material having desired characteristics, for example a shape memory alloy (e.g., nitinol or other nickel-titanium alloy, copper-based alloys, iron-based alloys, etc.), titanium, stainless steel, a biocompatible polymer, another metal or metal alloy, a ceramic, a composite or any combination thereof. The other implant may have any desired configuration to facilitate expansion, to resist contraction, and/or to impart a desired force on a structure during or after expansion. One or more expandable implants may be individually inserted into a bone, or may be joined or linked coaxially, in parallel, or in series to form a structure having desired characteristics. In some embodiments, the stents or other expandable implants may be resorbable. Further, the stents preferably should be constraint, particularly semi-constraint, in order to expand the stent where the stent should be deformed.

In some embodiments, a method of treating bone may include inserting inside a fractured or osteoporotic bone, for example a vertebrae, two or more coaxial stents that cooperate to augment a vertebral body. A bone cement or other filler may be added with or without the implanted devices to aid in stabilizing the bone and securing the implants in place within the bone. For example, bone grafting material, such as bone chips or demineralized bone may be added within the bone, and about the stent a plug of bone cement may be used to fix the stent in the vertebrae. In some embodiments, one or more additional implants may be used in combination with a stent, e.g., an expandable plug, an expandable bobbin, an expandable sheet metal implant, a chain, a pedicle screw, and the like, for example to expand the stent and/or provide additional augmentation.

In one embodiment an apparatus for osteopathic augmentation includes a first expandable implant having a first configuration and a second configuration, the expandable implant capable of undergoing plastic deformation in its second configuration, and an expansion device being at least semi-constraint, wherein the implant surrounds at least a portion of the expansion device. The expansion device and the implant are configured and dimensioned for insertion into a region of bone through a cannula, where the implant is capable of sustaining between about 5 N and 300 N force applied to its perimeter.

In another embodiment, a method of augmenting a vertebral body includes providing a balloon catheter having a shaft with a lumen and a balloon portion operatively associated with the lumen, providing an expandable implant having a first implantable size and configuration capable of undergoing plastic deformation to a second expandable size larger than the implantable size and an expandable configuration different than the implantable configuration, the expandable implant mounted on the balloon portion of the balloon catheter, and inserting the balloon catheter with implant mounted thereon into the interior of a vertebral body so that the balloon portion and implant at least partially resides within the vertebral body. The method further includes expanding the balloon portion of the balloon catheter to change the implant to its expandable size and configuration, and removing at least the balloon shaft from the vertebral body.

In still another embodiment, a kit may comprise various combinations of components according to the present invention. A kit may include, for example, a cannula and one or more expandable implants. A kit may additionally include one or more balloons or other expandable members for imparting an expansion force to the one or more implants. A kit may additionally include a syringe or other apparatus for injecting a cement or other filler into a vertebral body. Optionally, one or more other implants or devices may be included in a kit.

### BRIEF DESCRIPTION OF THE DRAWINGS

The invention is explained in even greater detail and may be better understood by the following exemplary drawings, wherein like references numerals represent like elements. The drawings are merely exemplary to illustrate certain features that may be used singularly or in combination with other features and the present invention should not be limited to the embodiments shown.

FIG. 1 is an illustration of a spine having a vertical compression fracture in one vertebral body;

FIGS. 2A-D are illustrations of a prior art method for treating a vertical compression fracture;

FIG. 3 is an illustration depicting a prior art method of stenting a vertebral body;

FIG. 4 is a perspective view of an expandable implant according to an embodiment of the present invention;

FIG. 5 is a side view of the implant of FIG. 4, along with balloon device for expanding the implant;

FIG. 6 is a cross-sectional side view of a vertebra depicting a method of inserting a cannula through the pedicle of the vertebra;

FIG. 7 is a cross-sectional side view of the vertebra of FIG. 6, showing a balloon-tipped catheter carrying an expandable implant that is inserted through the cannula and into the vertebral body;

FIG. 8 is a cross-sectional side view of the vertebra of FIG. 6, showing a balloon and implant inserted over a guide wire;

FIG. 9A is a cross-sectional top view of a vertebra having implants inserted bilaterally into a vertebral body;

FIG. 9B is another cross-sectional top view of a vertebra having one or more implants inserted over a guide wire within the vertebral body;

FIG. 10 is a cross-sectional side view of the vertebra of FIG. 7, with the balloon inflated to expand the implant;

FIG. 11 is a cross-sectional side view of the vertebra of FIG. 10, showing the expanded implant within the vertebral body and the balloon removed;

FIG. 12 is a cross-sectional side view of the vertebra of FIG. 11, showing insertion of a balloon carrying a second implant;

FIG. 13 is a cross-sectional side view of the vertebra of FIG. 12, showing inflation of the balloon carrying the second implant;

FIG. 14 is a cross-sectional side view of the vertebra of FIG. 13, showing removal of the balloon;

FIG. 15 is a cross-sectional side view of the vertebra of FIG. 14, showing insertion of a third implant inside of the expanded second implant;

FIG. 16A-C are cross-sectional side view of a vertebra, showing insertion of bone cement prior to insertion of an expandable implant;

FIG. 17 is a cross-sectional side view of a vertebra showing a catheter having a lumen for injection of bone cement along with a balloon-implant;

FIG. 18 is a cross-sectional side view of a vertebra having an expanded implant and bone cement being inserted after removal of the balloon;

FIG. 19 is a cross-sectional side view of a vertebral showing a balloon filled with cement inside of an expanded implant;

FIGS. 20A and B are side view illustrations of an embodiment of an expandable implant before and after expansion, respectively;

FIG. 21 is a side view of another embodiment of an expandable implant;

FIG. 22 is a side view of another embodiment of an expandable implant;

FIGS. 23A and B are perspective views of an embodiment of an expandable sheet implant before expansion (left) and after expansion (right);

FIGS. 24 and B are cross-sectional end view of an implant assembly before and after expansion, respectively;

FIG. 25-C are cross-sectional end views of another embodiment of an implant assembly;

FIG. 26 is an end view of a ratchet mechanism of another embodiment of an implant assembly;

FIG. 27 is a side view of helical shaped implants according to another embodiment of the invention;

FIG. 28 is a cross-sectional side view of the helical implants of FIG. 27 inserted into a vertebral body;

FIG. 29A is a side view of two helical implants having converse windings;

FIG. 29B is a side view of the helical implants of FIG. 29A joined together;

FIGS. 30A-D are side views of implant assemblies incorporating expandable plugs for expanding the outer implants;

FIGS. 31A-C are cross-sectional side views showing use of another embodiment of an expandable implant assembly;

FIG. 32 is a cross-sectional side view of a another embodiment of an expandable implant assembly within a vertebral body; and

FIGS. 33A and B are perspective views of an expandable implant before (A) and after (B) expansion of an inner coiled member.

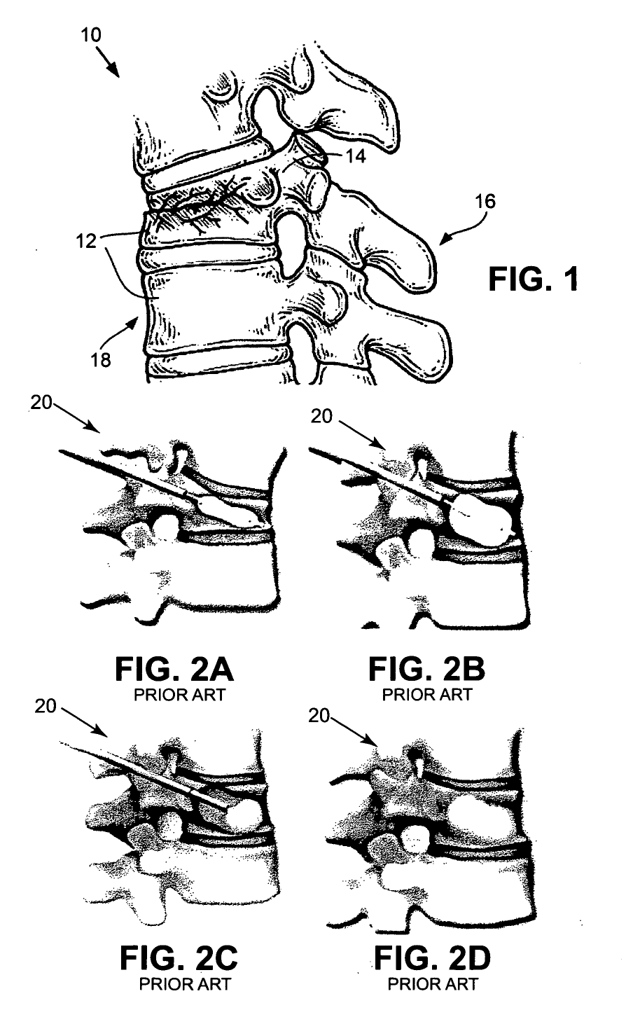

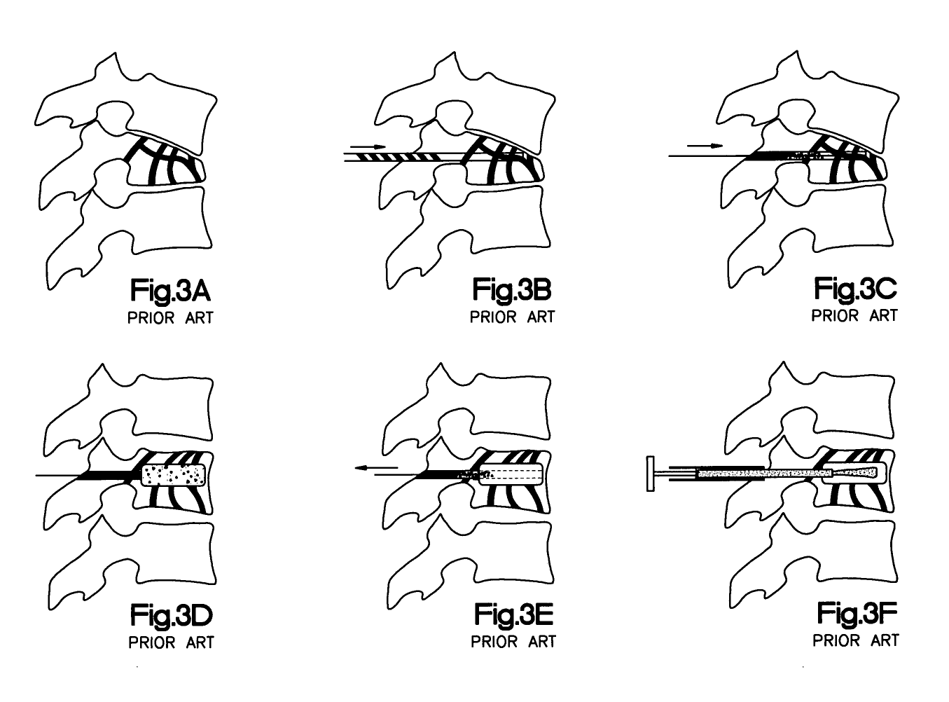

### DETAILED DESCRIPTION

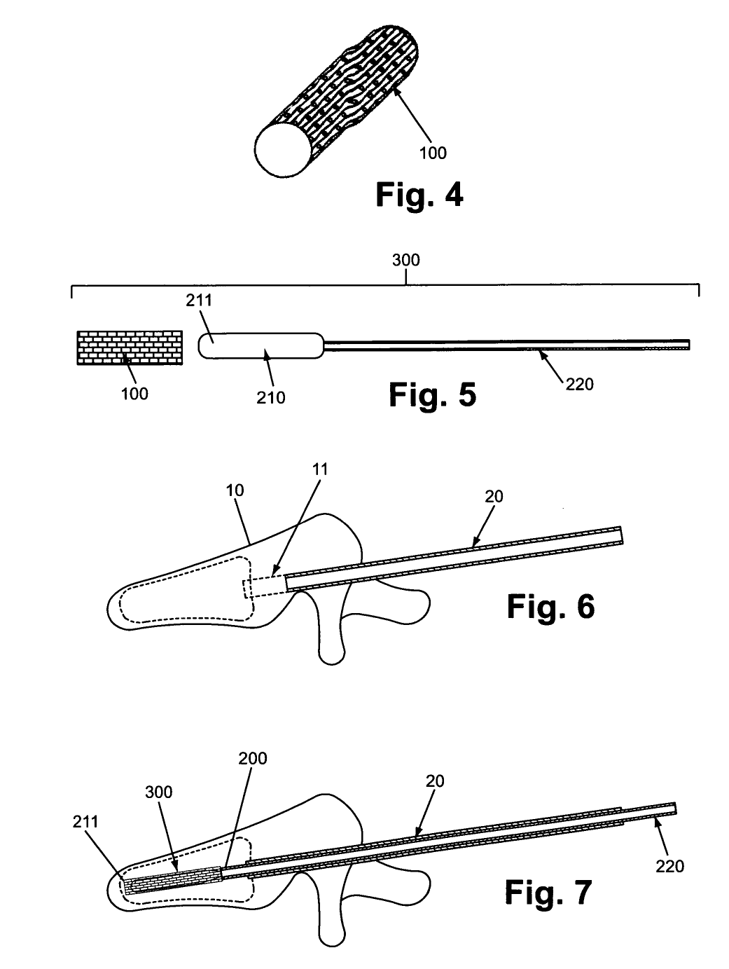

FIG. 4 shows a three dimensional view of an expandable implant 100 in an unexpanded state. Such an implant 100 may be inserted into a vertebral body (not shown) or other bone to repair damage to the bone, e.g., a spinal compression fracture. In some embodiments, the lordosis of the spine is reconstructed using a balloon catheter (e.g., such as described above for kyphoplasty) that carries one or more expandable implants that remain inside of the vertebral body and prevents the loss of reposition after removal of the balloon catheter or other device used for expanding the implant.

The implants are preferably expandable and resist collapsing forces, preferably forces, for example, between about 5N and about 300N. In some embodiments, the implants may have the form of a tube and may comprise one or more parts. Several implants may be inserted into each other to achieve a stable construct that can hold the interoperative compression forces acting on the vertebral body.

The implants may be made out of a biocompatible shape memory alloy, stainless steel, cobalt chromium alloy, titanium or alloy thereof, a polymer, tricalcium phosphate, or any other material having desired characteristics. In some embodiments, the implant may be covered or coated, for example with a biodegradable polymer.

In embodiments comprising a shape memory alloy (e.g., nitinol), implants may expand when heated to a temperature over an actuation temperature, for example as the shape memory alloy undergoes a phase transformation between a Martensite state (e.g., at a low temperature) state and an Austensite state (e.g., at a higher temperature). The actuation temperature of shape-memory alloy fibers within the implant may preferably be, for example, between about 28° C. and about 36° C. Alternatively, an implant mass may expand, contract, or otherwise change shape or configuration when it is activated by an energy source (e.g., an ultraviolet light, ultrasonic radiation, radio waves, heat, electric filed, or magnetic field).

An unexpanded implant may have any desired diameter that preferably fits through a lumen of a cannula and into a vertebral body. For example, in some embodiments, the diameter of an implant 100 of FIG. 4 before insertion may be between about 2 mm and 10 mm. In an expanded state, the diameter of implant 100 may be between about 15 mm and 25 mm. Of course, other sizes may be used without departing from the scope of the invention.

FIGS. 5 to 15 depict a method of using an expandable implant 100 to reposition and augment a collapsed vertebral body 10, for example to reposition the endplates of the vertebral body 10 and to hold the reposition after reconstruction of spinal lordosis. The method and implants may be used to reposition and augment other bones.

In particular, FIG. 5 shows an expandable implant dimensioned to fit around a balloon catheter assembly 200 or other device, also referred to herein as an “expansion device” for imparting an expansion force to the implant. The balloon catheter assembly 200 may comprise a balloon 210 and a catheter shaft 220 connected to one end of the balloon 210. Implant 100 disposed on a balloon 210 or other expansion device is referred to herein as a balloon-implant assembly 300. The implant 100, or a portion thereof, may in some embodiments be similar to a “stent” used to maintain patentcy of vessels during an angioplasty procedure. The stent, balloon, balloon-stent assembly, and/or balloon-catheter assembly may be coated with an adhesive, antibiotics, osteoinductive material, or osteoconductive material. After expansion, a user may actuate the adhesive by an energy source, e.g., heat, ultraviolet light, ultrasonic radiation, radio waves, electricity, magnetic field, etc.

The balloon 210 used as the expansion mechanism to expand the implant from its first insertion size to its second expanded size preferably is at least semi-constraint so that the balloon 210 can exert sufficient force on the areas of the implant desired to be expanded.

Preferably, the balloon 210 or expansion device is not compliant (not elastic) or semi-compliant like some balloon catheters on the market such as the balloon used in kyphoplasty where due to stent resistance it is believed the balloon would expand around the implant without providing necessary force to expand the stent. For example, if the balloon is too elastic it may expand anteriorly and posteriorly of the stent, and may even explode if the stent ends cut into the balloon. In this regard, it is preferable that the balloon be relatively inelastic so the force from the balloon can be directed to desirable areas of the stent. Alternatively, a relatively elastic balloon utilized with an outer jacket to restrain the elastic expansion may suffice as an expansion device.

The expandable stent is preferably undergoes plastic deformation when it expands to its second size so that when the balloon is deflated it returns its first insertion size, or thereabouts, allowing the user to remove the balloon, if necessary, leaving the expanded implant within the vertebral body.

FIG. 6 shows an access hole 11 drilled through a vertebral body 10 in a transpedicular approach. Such an access hole 11 may be formed through the outer cortical bone of the vertebra 10 by a drill, trocar, or other instrument (not shown). After the access hole is made through one or more pedicles of the vertebral body 10, a cannula 20 may be inserted into each access hole 11 in order to provide passage of expandable implant 100.

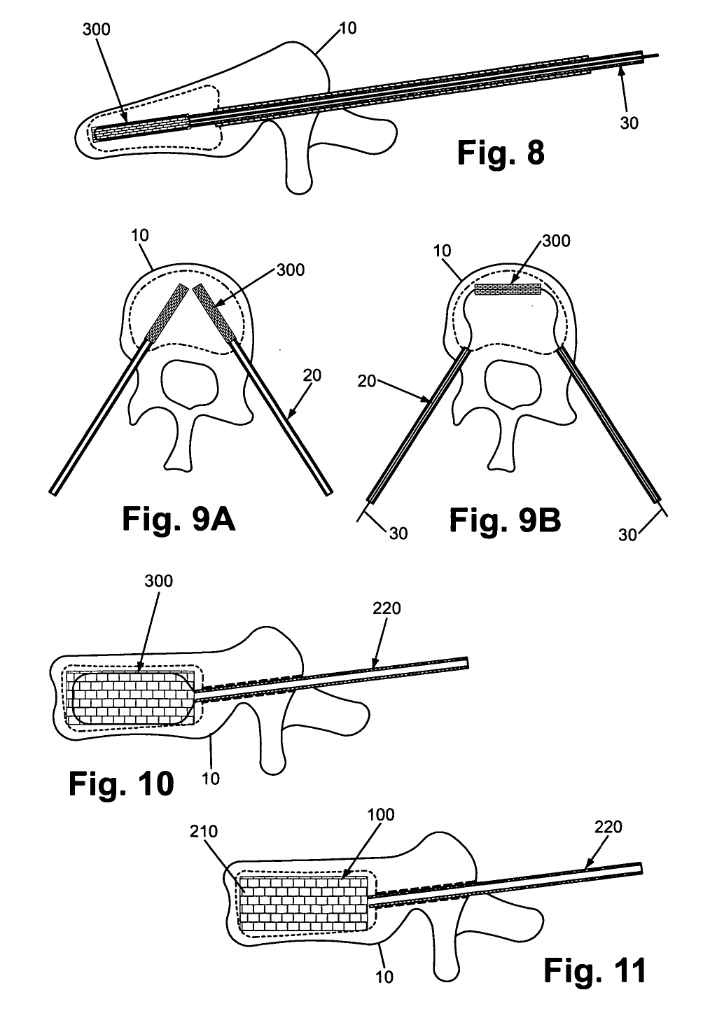

FIG. 7 shows a balloon catheter assembly 200 introduced by a single or bilateral transpedicular approach, with an expandable implant 100 disposed over the expandable balloon tip 211 of the catheter shaft 220. The insertion of the catheter 220 may take place with a guiding device 30, e.g., a guide wire as shown in FIGS. 8 and 9B. The guiding device 30 may be used to position the implant 100 in a desired location or orientation within the vertebral body 10. For example, FIGS. 9A and 9B show different possible positions of the balloon-implant assembly 300 (stent device). Both a cannula and a guide wire may be used together, or separately, to guide the balloon-implant assembly 300. Alternatively, neither the cannula or guide wire may be used to position the implant, rather the balloon-implant assembly 300 may be inserted down the passage way formed in the bone by a physician manipulating the catheter shaft 220 from outside the patient.

FIG. 10 depicts an exemplary method of repositioning and augmenting a vertebral body 10 using an expandable implant 100 by inflating the balloon 210 (e.g., with a radiolucent fluid), which imparts a radial force on the surrounding implant 100, thereby expanding the implant 100 to fill the space within the vertebral body 10 and restore the height of the vertebral body 10.

After the implant 100 is expanded to a desired diameter, the balloon 210 may be deflated and removed from the cannula 20, as shown for example in FIG. 11. The implant 100 is preferably configured to maintain its expanded dimension and to maintain the height of the vertebral body 10 even after the balloon 210 is removed. Alternatively, the balloon 210 and implant may detach from the catheter shaft 220 so that the balloon-implant assembly 300 remains in the bone.

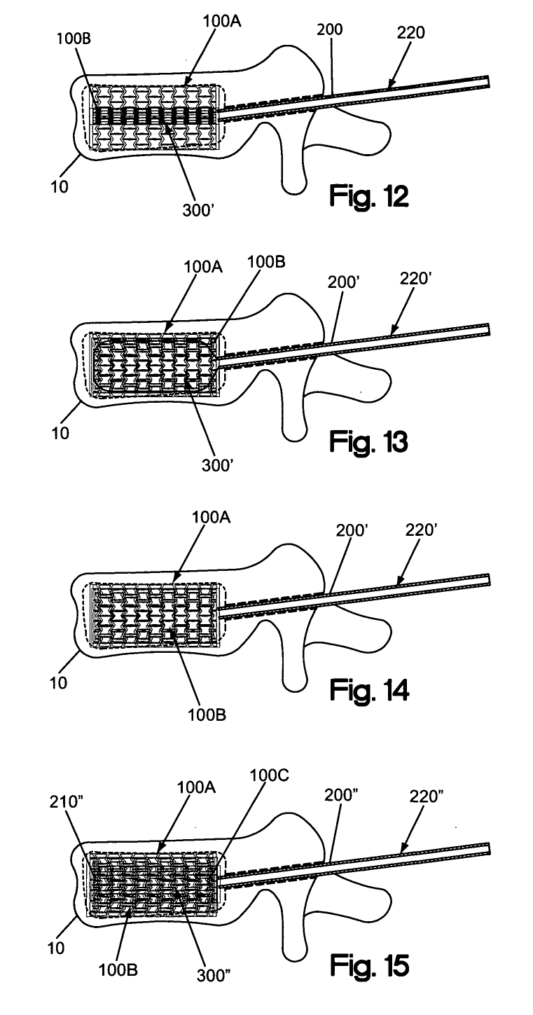

As shown in FIG. 12, a second expandable balloon-implant assembly 300′ (comprising balloon catheter assembly 200′ which includes catheter shaft 220′, and implant 100B) may be inserted into the vertebral body 10, e.g., within the lumen of the first expanded implant 100A to provide further support. After insertion, the inner balloon or other expansion device may be inflated or otherwise expanded as shown in FIG. 13 to expand the second implant 100B, e.g., such that it engages with the inner surface of the first implant 100A. The second implant 100B may be placed coaxially within the first implant 100A to increase resistance against collapse of the vertebral body 10. As with FIG. 11 above, after insertion of the second implant 100B, the interior expansion device may be removed as shown in FIG. 14. FIG. 15 depicts the insertion of a third balloon-implant assembly 300″ (comprising balloon catheter assembly 200″ which includes catheter shaft 220″, and implant 100C) coaxially arranged with the first and second implants. Any desired number of implants may be inserted within the first implant.

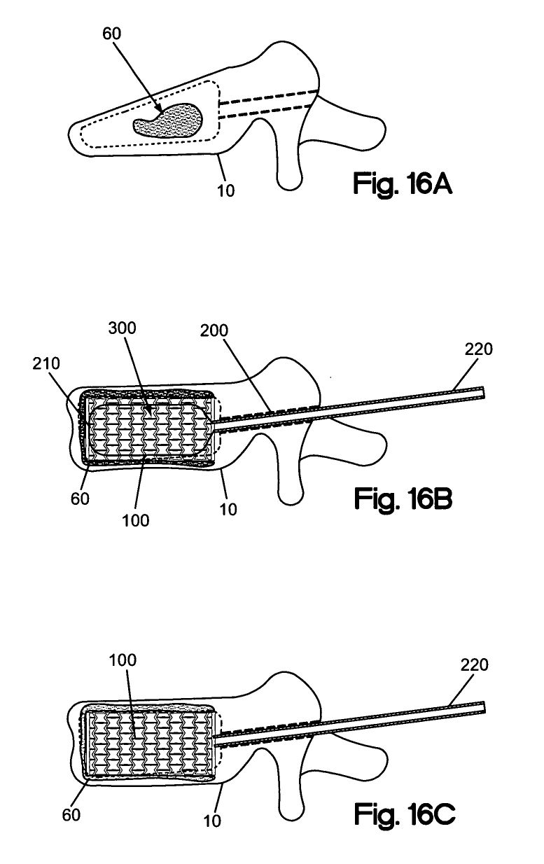

Bone cement, bone chips, or other filler 60 may be added to further augment a vertebral body 10, and to lock the one or more implants (stents) into place. The filler 60 may further comprise antibiotics, bone morphogenic protein (BMP), growth hormones, etc. Such bone cement or other filler 60 could be put into the vertebral body 10 before, during or after insertion of the implant. For example, in some embodiments, the cement or other filler 60 may be inserted into the vertebral body 10 before insertion of the implant or expansion of the implant, as shown in FIG. 16A, for example. FIG. 16B depicts an expanded balloon-implant assembly 300 in the vertebral body 10 such that the cement or other filler 60 is dispersed around the balloon 210 and the implant. FIG. 16C depicts the cement or other filler 60 dispersed around the expanded implant 100 after the balloon 210 has been removed. In other embodiments, bone cement or other filler 60 may be injected into the interior of the implant after its expansion and removal of the balloon catheter assembly. In other embodiments, bone cement or other filler 60 may be injected into the vertebral space during expansion of the implant. In still other embodiments, bone cement or other filler 60 may be injected into the balloon catheter assembly 200 to expand the balloon and consequently the implant. The catheter shaft 220 may thereafter be detached from the balloon and the balloon filed with filler as well as the expanded implant may remain in the bone. After the catheter shaft 220 is detached from the balloon, the passage way 225 of the catheter shaft 220 may be used to inject additional filler 60 or different filler 60′ in the bone, and to surround or assist in stabilizing the expanded implant.

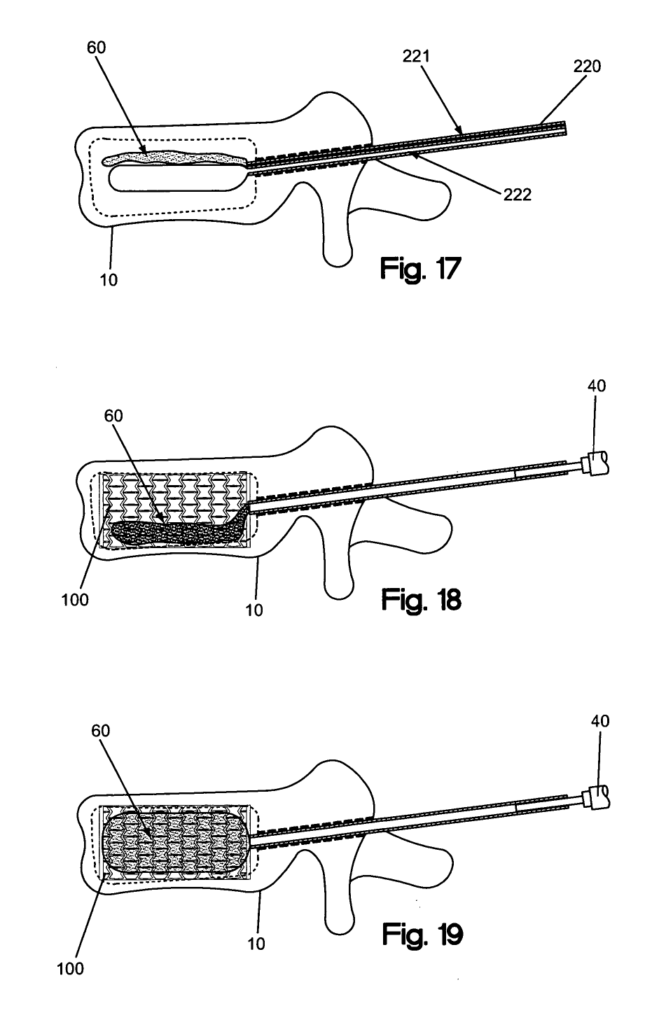

In some embodiments, a catheter may have multiple lumens, for example two lumens 221, 222 shown in FIG. 17, may be used to provide access to the vertebral body 10 and to provide a passage for injecting other materials into the bone. Bone cement or filler 60 may be inserted through lumen 221. The material inserted through lumen 221 may contact the expandable implant, the exterior surface of the balloon 210 or both. Material can be inserted through second lumen 222 before expansion of the balloon so that the material can be distributed within the bone void upon expansion of the balloon 210, the implant, or both.

Distributing material on the exterior of the balloon 210, implant, or balloon-implant combination, or within the bone void may assist in stabilizing the implant upon its expansion in the bone. In addition or alternatively, a material, such as a biocompatible polymer, can be inserted through lumen 221 into the bone void, or onto the exterior surface of the balloon 210, the implant 100 or both, and distributed in the bone void upon expansion of the balloon 210 to form an enclosure, or bag to prevent any balloon filler material from escaping the bone void. The biocompatible polymer can act to seal the otherwise porous cancellous bone to prevent any leakage of the balloon filler material. Cement or other filler 60 may also be inserted using, for example a syringe 40, after the removal of the balloon 210 (but with the implant remaining in the bone (FIG. 18)). In another embodiment, the balloon 210 may be filled directly with bone cement or another filler 60, as shown in FIG. 19. For example, such a balloon 210 may be expanded within the vertebral body 10 until the balloon 210 bursts. Alternatively, the balloon 210 may be filled with and expanded by bone cement or other filler 60 and the expanded balloon 210 thereafter detached from the catheter shaft 220 so that it remains in the patient. The balloon may be formed of a biosorbable material so that it will be absorbed by the body over time. Also, the balloon 210 may also be perforated with holes as described by Publication WO 03/099171 A1 (PCT/EP03/05407) to Grönemeyer et al. The balloon 210 may be filled with hardenable cement that will harden in situ. After the cement has hardened in the balloon 210, the balloon 210 may be detached from the catheter shaft 220.

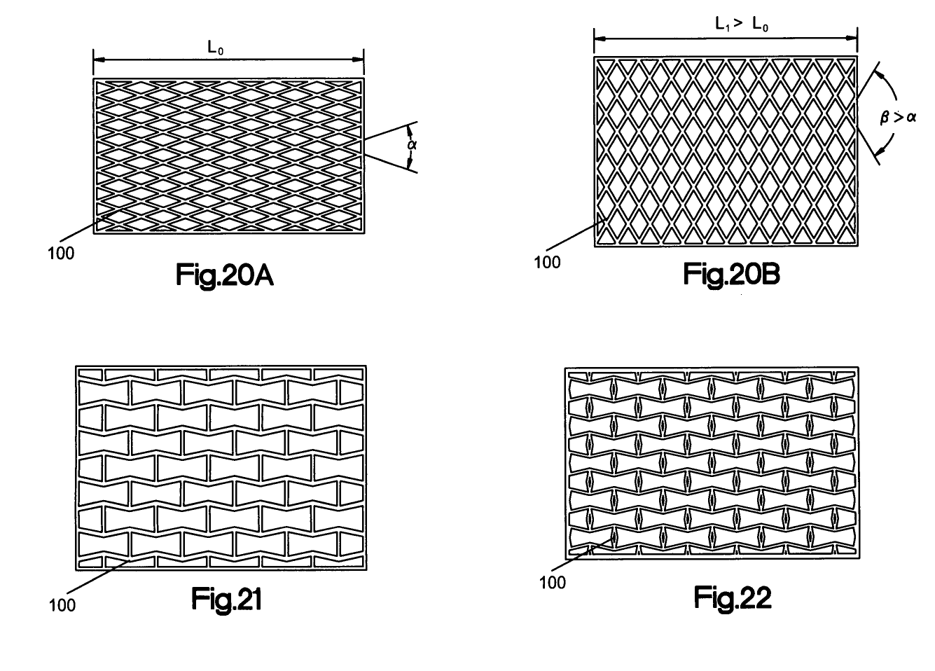

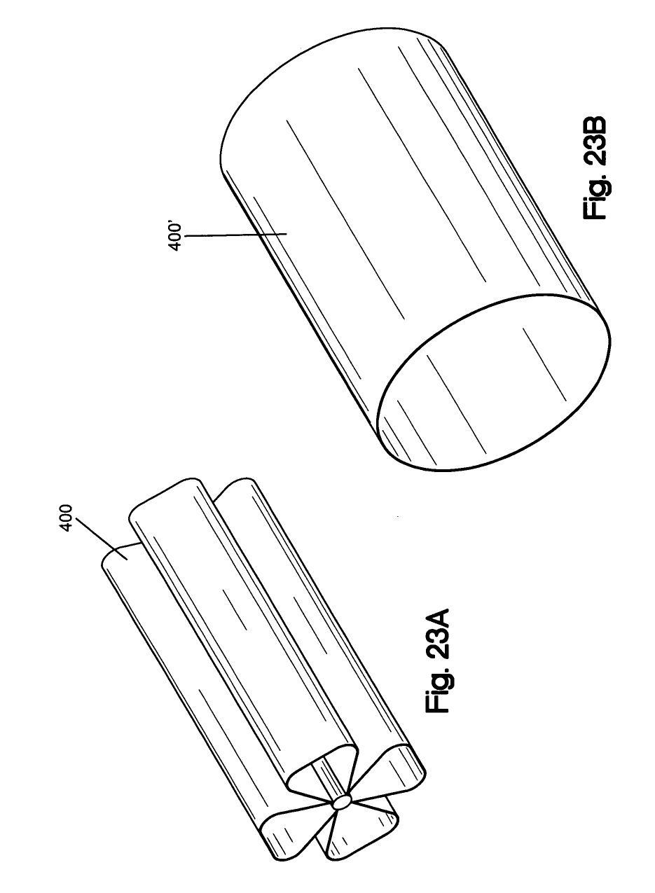

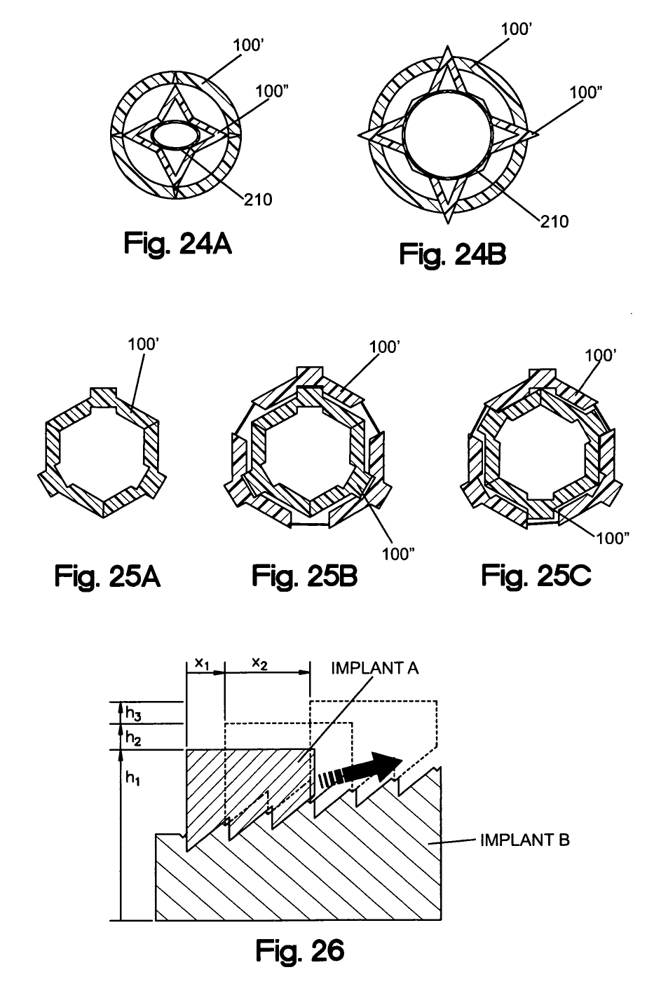

FIGS. 20A to 33 depict implants, or stents, with different configurations and/or patterns, including a continuous cylinder with perforations. It is important that an expanded implant resists the pressure exerted by the environment that is inserted. For example, if positioned within the vertebral body the expandable implant preferably is capable of withstanding the force that will b exerted on the vertebral body during a patient's routine activity following insertion of the implant. Preferably the implant will not shorten significantly after expansion (e.g., deviation between L1-L0 needs to be small). FIGS. 20 to 22 show various mesh-like patterns that may be used by the expandable implants. In FIG. 20A, the mesh-like pattern of an unexpanded implant may form angles α. When the implant is expanded (FIG. 20B), the mesh-like pattern forms angles β, where, β is greater than α. FIGS. 23A and B show an expandable implant formed of folded sheet metal for insertion. FIG. 23A depicts the implant 400 before expansion and FIG. 23B depicts the implant 400′ after expansion. In preferred embodiments, such a folded sheet metal insertion device is made from a shape memory alloy (e.g., nitinol).

One skilled in the art will appreciate that the geometry of the implants can be chosen in a way that several implants may wedge into each other when they are expanded. Thus, the implants may resist higher compression forces when they are in their inflated state. FIGS. 24-26, for example, show different principles of how the cross-section of the implants/stents (100′ and 100″) may be configured to wedge the implants into each other. In FIG. 26, implant A, the inner implant, and B, the outer implant, may be shaped like a cone, with a serrated (teethed) surface. By pulling implant B from “x1” to “x2”, the outer diameter may be expanded from “h1” to “h3”. Due to the serrated surface, B, the outer implant keeps the expanded shape.

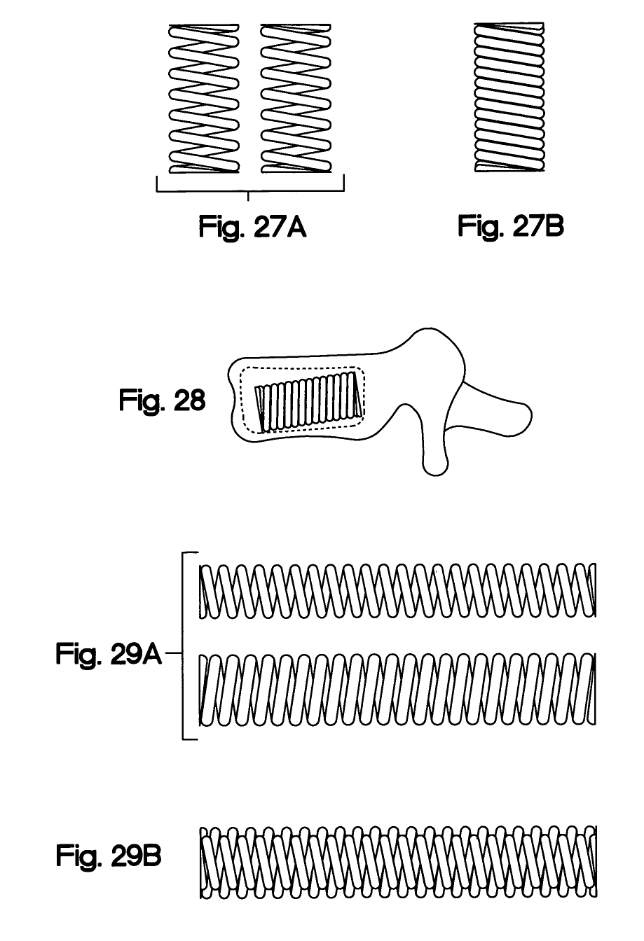

As shown in FIGS. 27-29, the one or more implants may have a helical configuration. Several helical implants may be screwed into each other, such that a stable implant is achieved. In particular FIGS. 27 and 28 depict helical implants having coils in the same direction. FIGS. 29A and B show helical implants having coils in different directions. Regardless of the direction of the coils, the helical implants are configured to screw into each other, thereby strengthening the implant.

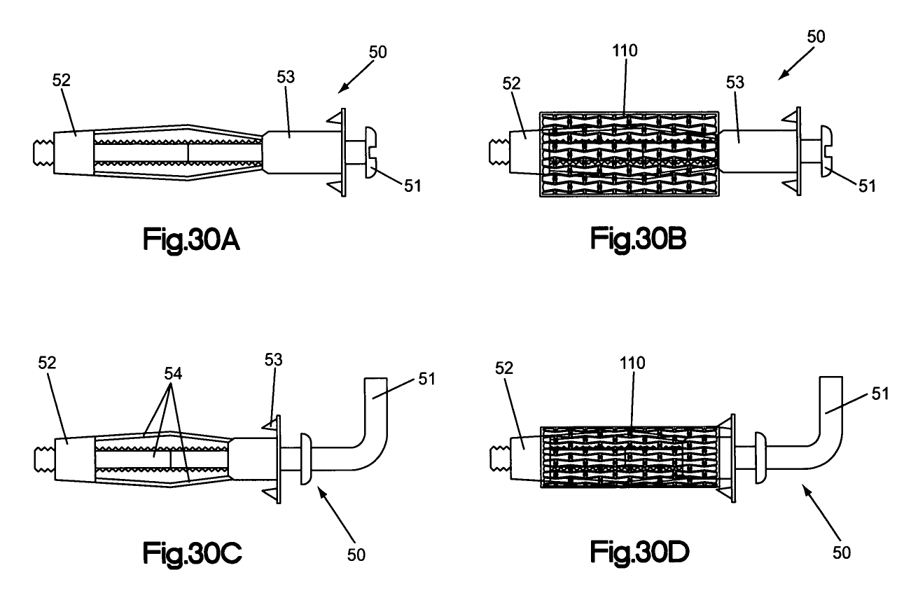

FIGS. 30-33 show alternatives to a balloon 210 for providing the expansion force to expand an implant 100. For example, FIGS. 30A-D show another embodiment of an implant 110, which uses an expandable plug 50 instead of a balloon 210 to expand the implant 110. The plug 50 may consist of a expansion mechanism 51 such as a screw (FIGS. 30A and C) or a L-handle (FIGS. 30B and D). Other configurations of the expansion mechanism 51 are contemplated. The plug 50 may also have a distal end 52, a proximal end 53 and a plurality of expansion supports 54 connecting the distal end 52 with the proximal end 53. The expansion mechanism 51 is threaded onto both the distal end 52 and proximal end 53 such that as a user rotates the expansion mechanism 51, the distal end 52 moves towards the proximal end 53 causing the plurality of expansion supports 54 to expand outward thereby expanding implant 110. The user continues rotating the expansion mechanism 51 until the implant 110 reaches its desired diameter. The implant 110 and plug 50 may be inserted into the vertebral body (not shown) in the unexpanded state as illustrated in FIGS. 30A and B. Thereafter, the implant 110 can be expanded as shown by FIGS. 30C and D by turning the expansion mechanism 51, (e.g., for example the screw) of the plug 50. After expansion of the implant 110, the plug 50 may be removed. In other embodiments, the plug 50 may be left in the bone.

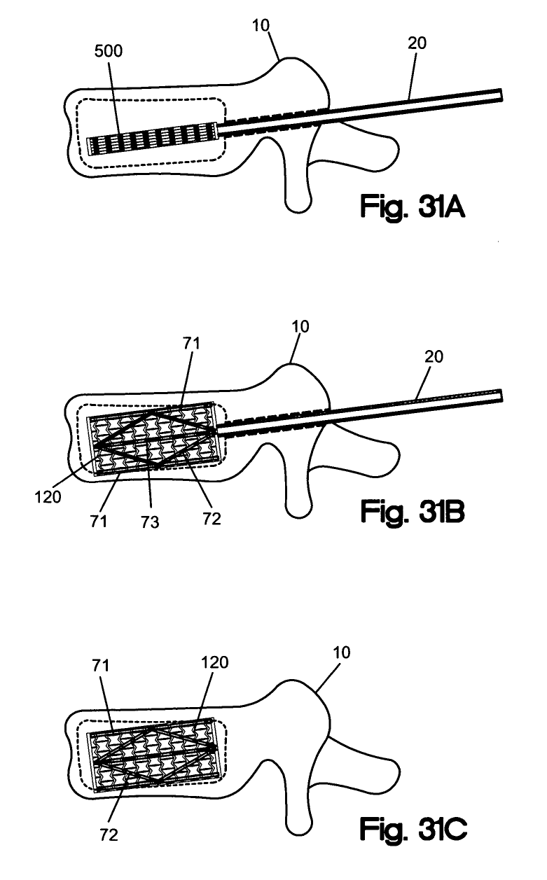

FIGS. 31A-C show another mechanism for expanding the implant. In particular, the implant may be expanded using a jack mechanism 70, for example working in a manner similar to a car jack. As shown in FIG. 31A, an implant assembly 500 may be inserted into the vertebral body 10 through a cannula 20. The implant assembly may consist of an implant 120 and the jack mechanism 70. The jack mechanism 70 may consist of a plurality of expansion members 71, preferably at least two expansion members 71, a plurality of diagonal braces 72, and a drive mechanism 73. Operation of the drive mechanism 73 causes the plurality of diagonal braces 72 to extend outward from the drive mechanism 73 pushing the plurality of expansion members 71 similarly outward from the drive mechanism 73. The outward movement of the expansion members 71 causes the implant 120 to expand (FIG. 31B) until the desired diameter of the implant 120 is achieved. After the implant 120 is expanded to the desired diameter, the drive mechanism is removed leaving the expansion members 71 and diagonal braces 72 (FIG. 31C).

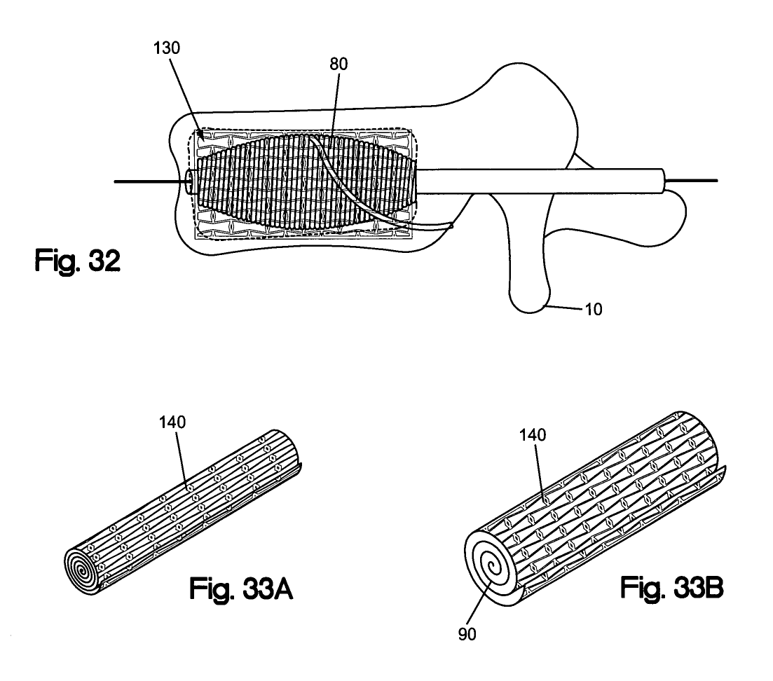

FIG. 32 shows the use of a bobbin complex 80 for expanding the implant 130 and thus the height of the vertebral body. Such a bobbin complex 80 is described in more detail, for example, in U.S. patent application Ser. No. 11/471,169, the disclosure of which is incorporated by reference herein. FIG. 33 shows a method of expanding implant 140 using a sheet metal coil 90, for example as described also in U.S. patent application Ser. No. 11/471,169.

Although the apparatus and methods described herein thus far have been described in the context of repositioning and augmenting vertebrae in the context of vertebral compression fractures and deformations in spinal curvature, various other uses and methods are envisioned. For example, in some embodiments, one or more implants comprising expandable stents may be used to reposition and/or augment other damaged bone regions such as a fractured or weak proximal femur.

In such embodiments, for example, one or more implants may be inserted into a head of a femur, e.g., through a cannula or other introducer, or for tibia plateau fracture repositioning. Once inserted, the implants may expand and compact material within the head of the femur and provide solid support to augment the head. In some embodiments, the implant may comprise a shape memory alloy and expand or otherwise change its configuration after insertion (e.g., after heating to a temperature above an activation temperature). A bone cement or other filler may also be used to aid augmentation. In other embodiments, another implant such as a screw or other device may be inserted in addition to or instead of one or more implants.

In some embodiments, the implants and methods described herein may be used in conjunction with other apparatus and methods to restore lordosis and augment a vertebral body. For example, one or more expandable implants may be used in conjunction with known procedures, e.g., a balloon kyphoplasty, which may be used to begin repositioning of a vertebral body and/or create a space within the body for the implant. In other embodiments, one or more implants described herein may be used in conjunction with other tools or devices, e.g., an external fixation apparatus for helping to manipulate or fix the vertebrae or other bones in a desired position.

In another embodiment, a kit may comprise various combinations of components according to the present invention. A kit may include, for example, a cannula and one or more expandable implants. A kit may additionally include one or more balloons, balloon catheters or other expandable members for imparting an expansion force to the one or more implants. A kit may additionally include a syringe or other apparatus for injecting a cement or other filler into a vertebral body, or into the balloon or balloon catheter. Optionally, one or more other implants or devices may be included in a kit. One skilled in the art will appreciate that various other combinations of devices, components and assemblies can be made and are intended to fall within the scope of the present invention.

In other embodiments, various minimally invasive implants and methods for alleviating discomfort associated with the spinal column may employ an expandable implant having one or more of the features described herein. For example, an expandable implant or other implant comprising a shape-memory alloy may be implanted between spinous processes of adjacent vertebrae, and the implant may be expanded or otherwise altered in its configuration to distract the spinal processes and alleviate pain and other problems caused for example by spinal stenosis, facet arthropathy, and the like. For example, augmentation systems described herein may be used instead of or in addition to expandable interspinous process apparatus and methods described in U.S. Patent Publication No. 2004/018128 and U.S. Pat. No. 6,419,676 to Zucherman et al.

While the foregoing description and drawings represent the preferred embodiments of the present invention, it will be understood that various additions, modifications and substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. The presently disclosed embodiments are therefore to be considered in all respects as illustrative and not restrictive, the scope of the invention being indicated by the appended claims, and not limited to the foregoing description.

All references cited herein are incorporated herein by reference in their entirety and for all purposes to the same extent as if each individual publication or patent or patent application was specifically and individually indicated to be incorporated by reference in its entirety for all purposes.

What is claimed is:

1. An apparatus for osteopathic augmentation comprising:
a first expandable implant having a first configuration and a second configuration, the expandable implant capable of undergoing plastic deformation in its second configuration; and
an expansion device being at least semi-constraint, wherein the implant surrounds at least a portion of the expansion device,
wherein the expansion device and the implant are configured and dimensioned for insertion into a region of bone through a cannula, and
wherein the implant is capable of sustaining between about 5 N and 300 N force applied to its perimeter.

2. An apparatus according to claim 1, wherein the implant comprises a shape memory alloy, titanium, a stainless steel, a metal alloy, a resorbable polymer, a non-resorbable polymer, a ceramic or a combination thereof.

3. An apparatus according to claim 2, wherein the shape memory alloy comprises nitinol.

4. An apparatus according to claim 1, wherein the first expandable implant is a stent configured as a continuous cylinder with perforations.

5. An apparatus according to claim 1, further comprising a second expandable implant configured for coaxial insertion within the first expandable implant.

6. An apparatus according to claim 1, wherein the expansion device is capable of providing about 300 N force.

7. An apparatus according to claim 1, wherein said region of bone may include a tibia plateau fracture reposition.

8. An apparatus according to claim 1, wherein the expansion device is a balloon catheter having a shaft with an inflation lumen and a balloon portion associated with the distal end of the shaft.

9. An apparatus according to claim 8, wherein the balloon is detachable from the shaft.

10. An apparatus according to claim 1, wherein the expandable implant has an insertable size and configuration such that it is disposable upon the outside of a balloon catheter and insertable through a cannula and the expandable implant has an expandable configuration wherein the implant is plastically deformed to a larger circumferential size than its inflatable size.

11. An apparatus according to claim 1, wherein the expansion device may be coated with an adhesive, and
wherein after expansion, a user may actuate the adhesive by an energy source, such as heat, ultraviolet light, ultrasonic radiation, radio waves, electricity, or a magnetic field.

12. A method of augmenting a vertebral body comprising:
a) providing a balloon catheter having a shaft with a lumen and a balloon portion operatively associated with the lumen;
b) providing an expandable implant having a first implantable size and configuration capable of undergoing plastic deformation to a second expandable size larger than the implantable size and an expandable configuration different than the implantable configuration, the expandable implant mounted on the balloon portion of the balloon catheter;
c) inserting the balloon catheter with implant mounted thereon into the interior of a vertebral body so that the balloon portion and implant at least partially resides within the vertebral body;
d) expanding the balloon portion of the balloon catheter to change the implant to its expandable size and configuration; and
e) removing at least the balloon shaft from the vertebral body.

13. A method according to claim 12, wherein before the step of removing at least the balloon shaft, decreasing the balloon portion after the implant has been changed to its expandable size.

14. A method according to claim 12 further comprising removing the balloon catheter from the vertebral body.

15. A method according to claim 12 wherein the balloon portion of the balloon catheter is removable from the shaft of the balloon catheter and wherein the method further comprises detaching the balloon portion from the shaft and removing only the catheter shaft from the vertebral body.

16. A method according to claim 12 further comprising inserting material comprising bone cement through the lumen expanding the balloon portion.

17. A method according to claim 16 further comprising expanding the balloon portion with a flowable bone cement and permitting the bone cement to harden in situ in the vertebral body.

18. A method according to claim 12 further comprising inserting the bone cement into the vertebral body before the balloon catheter is inserted into the vertebral body.

19. A method according to claim 12 further comprising inserting bone cement into the interior of the expandable implant.

20. A method according to claim 12 further comprising inserting bone cement into the interior of the expandable implant after the balloon portion of the balloon catheter has been removed from the inside of the expandable implant.

21. A method according to claim 12 further comprising:
a) providing a cannula having a longitudinal passageway therethrough;
b) inserting the cannula into the vertebrae such that the passageway communicates with the interior of the vertebral body and the outside of the patient; and
c) removing the cannula from the vertebral body.

22. A method according to claim 12 wherein the shaft of the balloon catheter has more than one lumen communicating with the exterior of the balloon such that bone cement may be inserting through a second lumen to the exterior of the balloon coating the exterior of the balloon and implant with bone cement.
